(19) United States
(12) Patent Application Publication
(10) Pub. No.: US 2018/0310832 A1
(43) Pub. Date: Nov. 1, 2018

(54) FLEXIBLE TUBING FOR A PRESSURE MONITORING SYSTEM

(71) Applicant: Edwards Lifesciences Corporation, Irvine, CA (US)

(72) Inventors: Brian Patrick Murphy, Costa Mesa, CA (US); Jason A. Wine, Placentia, CA (US)

(21) Appl. No.: 15/960,160

(22) Filed: Apr. 23, 2018

Related U.S. Application Data
(60) Provisional application No. 62/607,227, filed on Dec. 18, 2017.
(60) Provisional application No. 62/490,724, filed on Apr. 27, 2017.

(51) Int. Cl. A61B 5/023; A61B 5/03

(52) U.S. Cl. CPC A61B 5/023; A61B 5/72; A61B 5/031

(57) ABSTRACT

Disclosed is a pressure monitoring system for a patient that may include a measurement site, a tube, and a pressure transducer, in which, the tube is coupled between the measurement site of the patient and the pressure transducer and the tube contains a fluid for pressure measurement by the pressure transducer from the measurement site. The tube may comprise: a first tubing portion that includes an outer diameter and a plurality of inner diameters and that has a first tensile modulus, in which, the plurality of inner diameters of the first tubing portion form a plurality of fluid paths to contain the fluid; and, a second tubing portion, in which, the first and second tubing portions are coupled together to form the tube and the second tubing portion extends longitudinally with the first tubing portion in a continuous or intermittent manner.

### CROSS-REFERENCE TO RELATED APPLICATIONS

This application claims the benefit of U.S. Application No. 62/607,227, filed Dec. 18, 2017, and U.S. Application No. 62/490,724, filed Apr. 27, 2017, incorporated herein by reference.

### BACKGROUND

### Field

The present invention relates to flexible tubing for a pressure monitoring system.

### Relevant Background

Blood pressure measurement systems often utilize pressure transducers (PTs) that are used to monitor blood pressure signals in a patient's vein or artery. PTs can also be used to monitor intracranial pressure as well as a wide variety of other types of pressure measurements. In particular, electrical pressure signals generated by PTs may be used for a number of monitoring and diagnostic applications and are often connected to a patient monitor to display graphical depictions of the signals generated, such as, pressure vs. time, etc. A PT is typically mounted near the patient and connected to the patient's vein, artery, cranium, or other part of the body via a catheter and a fluid-filled tube and to the patient monitor. Oftentimes, the PTs may be disposable pressure transducers (DPTs)). Patient monitors may employ sophisticated algorithms to derive volumetric and hemodynamic parameters from the pressure signal.

In particular, the pressure signal is generated and transmitted from the measurement site (e.g., vein, artery, etc.) via the catheter and the fluid-filled tube as fluid pressure to the pressure transducer (PT) where it may be converted to an electrical pressure signal outputted to the patient monitor. The PT is typically located in a plastic enclosure that ensures connectivity to the fluid-filled catheter-tubing system on one side and the patient monitor on the other side. The term PT or DPT usually refers to the system that includes the enclosure housing the pressure transducer, the transducer itself, and respective connectors. Therefore, as has been described, in these type of pressure measurement systems, a pressure transducer (PT) may be connected to a patient's artery or vein through a fluid column contained in tubing on one side and may output electrical pressure signals from the PT on the other side to the patient monitor.

Longer length tubing has been found to be desirable for ease of use and flexibility in connecting the patient to the pressure transducer system. However, it has been found that when the tube is of longer length, the fluid/tubing combination can resonate at frequencies relevant to those contained within the pressure waveform being measured and may cause errors.

One method to address this problem is to increase the natural frequency of the fluid/tubing combination. Significantly stiffer tubing may be utilized to accomplish this. However, highly flexible tubing is typically preferred for ease of use and handling and stiffer tubing is more difficult for use by health care providers. As has been described, longer length tubing is desirable for flexibility in connecting the patient to the pressure transducer system, but, as has been previously described, longer length tubing may provide frequency related errors. Accordingly, an implementation to provide both length and flexibility to tubing without frequency related errors is sought after.

### SUMMARY

Embodiments of the invention may relate to a pressure monitoring system for a patient. The pressure monitoring system may include a measurement site, a tube, and a pressure transducer, in which, the tube is coupled between the measurement site of the patient and the pressure transducer and the tube contains a fluid for pressure measurement by the pressure transducer from the measurement site. The tube may comprise: a first tubing portion that includes an outer diameter and a plurality of inner diameters and that has a first tensile modulus, in which, the plurality of inner diameters of the first tubing portion form a plurality of fluid paths to contain the fluid; and, a second tubing portion, in which, the first and second tubing portions are coupled together to form the tube and the second tubing portion extends longitudinally with the first tubing portion in a continuous or intermittent manner. The second tubing portion may have a second tensile modulus that is greater than the first tensile modulus of the first tubing portion.

### BRIEF DESCRIPTION OF THE DRAWINGS

FIG. 1 is a diagram illustrating an example environment in which embodiments of the invention may be practiced.

FIGS. 2A-2C are cross-sectional views of a tube having first and second tubing portions coupled together to form the tube, according to embodiments of the invention.

FIGS. 3A-3C are cross-sectional views of a tube having first and second tubing portions coupled together to form the tube, according to other embodiments of the invention.

FIGS. 4A-4B are cross-sectional views of a tube having first and second tubing portions coupled together to form the tube, according to other embodiments of the invention.

FIGS. 5A-5C are cross-sectional views of a tube having first and second tubing portions coupled together to form the tube, according to other embodiments of the invention.

FIGS. 6A-6B are cross-sectional views of a tube having first and second tubing portions coupled together to form the tube, according to other embodiments of the invention.

FIGS. 7A-7B are cross-sectional views of a tube having first and second tubing portions coupled together to form the tube, according to other embodiments of the invention.

FIGS. 8A-8B are cross-sectional views of a tube having first and second tubing portions coupled together to form the tube, according to other embodiments of the invention.

### DETAILED DESCRIPTION

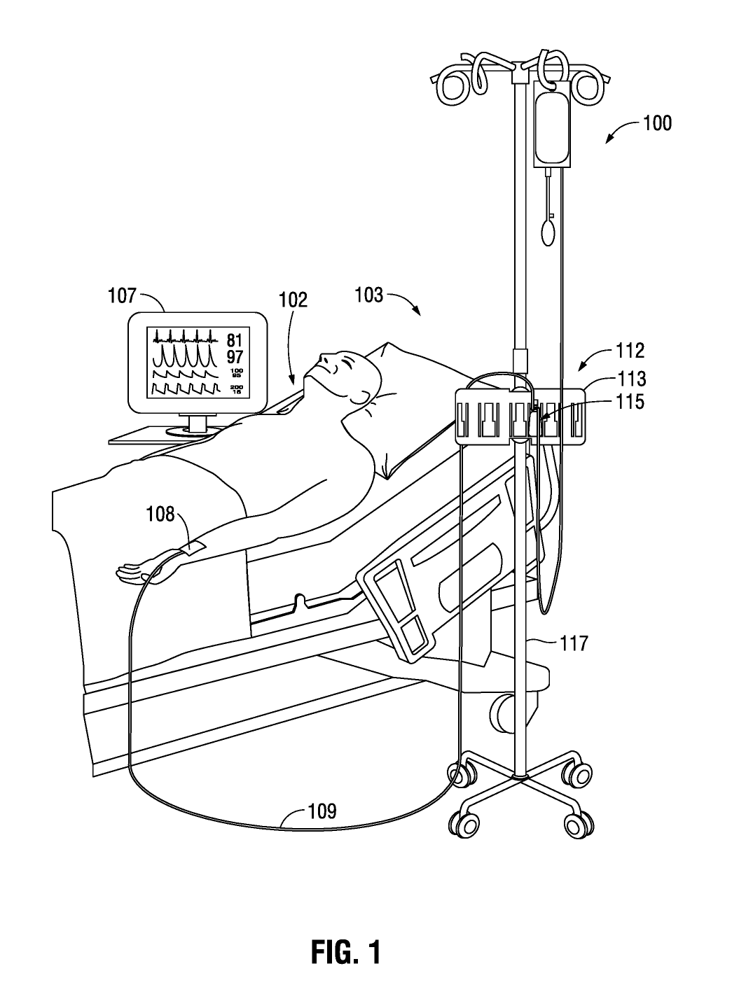

FIG. 1 is a diagram illustrating an example environment 100 in which embodiments of the invention may be practiced. In this example, a pressure monitoring system 103 for a patient 102 may be utilized that includes a measurement site 108, a tube 109, and a pressure transducer (PT) 115, in which, the tube 109 is coupled between the measurement site 108 of the patient 102 and the pressure transducer (PT) 115. Patient 102 may be, as an example, lying on a bed.

In this example environment 100, the blood pressure measurement system may utilize a pressure transducer (PT) 115 to monitor the blood pressure signals in the patient's 102 vein or artery. In particular, electrical pressure signals generated by the PT 115 may be used for a number of monitoring and diagnostic applications and may be connected to a patient monitor 107 to display graphical depictions of the signals generated, such as, pressure vs. time, etc. As can be seen on example patient monitor 107 such items as blood pressure, heart rate, etc., may be displayed.

A catheter may be connected to patient's 102 vein or artery at measurement site 108. It should be appreciated that this is just an example of measurement site at a patient's wrist and the measurement site 108 may be at any suitable patient location. Further, the pressure signal is generated and transmitted from the measurement site 108 (e.g., vein, artery, etc.) via the catheter and the fluid-filled tube 109 as fluid pressure to the pressure transducer (PT) 115 where it may be converted to an electrical pressure signal and outputted to the patient monitor 107. Thus, PT 115 may be connected to a patient's artery or vein at measurement site 108 through a fluid column contained in the tubing 109 on one side and may output electrical pressure signals from the PT 115 on the other side to patient monitor 107. Oftentimes, the PT may be a disposable pressure transducer (DPT).

As one example, a pressure transducer (PT) 115 may be connected to a plate 113 of a sensor holding apparatus 112 that is designed for holding various different types of transducers, sensors, medical devices, etc. As can be in seen FIG. 1, sensor holding apparatus 112 may be approximately rectangular cuboid shaped including six sides and may have a front plate 113 that may have various sensor holders for holding various types of sensors, transducers, medical devices, etc. Sensors that may be placed into the sensor holders may include, for example, PT 115. PT 115 may be considered to be a system that includes the enclosure housing for the pressure transducer, the transducer itself, and respective connectors. Further, the sensor holding apparatus 112 for holding PT 115, as well, as other types of sensors, transducers, medical devices, etc., may be mounted to a rolling IV stand 117 that further may be used to mount an IV bag mounted on the top of the stand to provide fluids. In particular, it should be appreciated that PT 115 may be located in a plastic enclosure that is mounted to a sensor holder of the sensor holding apparatus 112 that ensures connectivity to the fluid-filled tube 109 and catheter measurement site 108 on one side and the patient monitoring device 107 on the other side. In this way, PT 115 may be connected to a patient's artery or vein through a fluid column contained in tubing 109 on one side and may output electrical pressure signals from PT 115 on the other side to the patient monitor 107 for display.

It should be appreciated that a wide variety of other types of sensors, transducers, medical devices, etc., may be mounted to the sensor holders of sensor holding apparatus 112, such as, various other types of DPTs that may be for the measuring of Pulmonary Artery Pressure (PAP), Central Venous Pressure (CVP), Arterial Pressure (AP), etc.

By utilizing this system, PT 115 by measuring the pressure differences of the fluid in tube 109 may generate electrical output signals that corresponds to the patient's 102 blood pressure as measured at measurement site 108 and this corresponding electrical signal generated by PT 115 may be used for a number of monitoring diagnostic applications and, in particular, may be connected to patient monitoring device 107 to display a graphical depiction of blood pressure vs. time, as well as other types of data. It should be appreciated that a wide variety of other types of physiological data may be measured and displayed utilizing a suitable medical sensor device located at the sensor holding apparatus 112, and these are merely examples.

Embodiments of the invention may relate to a two part co-extruded tube, such as, tube 109, whose primary material (e.g., a polyvinyl chloride (PVC)) may have a first tensile modulus (e.g., relatively low—such as hardness approximately between 80 A-110 A), and may further utilize a secondary material that has a significantly higher tensile modulus (e.g., a hardness approximately 60 D). In some embodiments, the primary material may be an ethylene vinyl acetate (EVA) material. This secondary material may be co-extruded with the primary material somewhere between the inner diameter and the outer diameter of the tubing of tube 109. The secondary material may be routed longitudinally within the tubing of tube 109, forming veins of stiffening material. These veins may be positioned so as not to significantly increase the bending modulus of the tube 109 but may increase the tensile modulus of the tube 109.

By increasing the tubing tensile modulus in this way, the stiffness of tube 109 increases and the natural frequency of tube 109 increases, resulting in a higher fidelity pressure monitoring signal, while maintaining equivalent tubing flexibility to that of current pressure monitoring tubing. Therefore, the natural frequency increases, allowing for longer tubing, while maintaining tubing flexibility, allowing for ease of use by health care professionals.

As has been described with reference to FIG. 1, a pressure monitoring system 100 may include a measurement site 108, a tube 109, and pressure transducer 115 that may be held by a sensor holding apparatus 112, as previously described. Tube 109 is coupled between the measurement site 108 of the patient 102 and the pressure transducer 115. As has been described, the pressure signal is generated and transmitted from the measurement site 108 (e.g., vein, artery, etc.) via the catheter and the fluid-filled tube 109 as fluid pressure to the pressure transducer (PT) 115 where it may be converted to an electrical pressure signal and outputted to the patient monitor 107. Thus, tube 109 may contain a fluid for pressure measurement by the pressure transducer 115 from the measurement site 108.

As will be described, in one embodiment, tube 109 may include a first tubing portion that includes an outer diameter and an inner diameter and that has a first tensile modulus. Further, a second tubing portion may be coupled with the first tubing potion to form the tube 109. In this embodiment, the second tubing portion may extend longitudinally with the first tubing portion in a continuous manner or an intermittent manner As will be described, in various embodiments, the second tubing portion may have a second tensile modulus that is greater than the first tensile modulus of the first tubing portion and the second tubing portion may be located adjacent to the outer diameter of the first tubing portion or the second tubing portion may be located between the outer diameter and inner diameter of the first tubing portion.

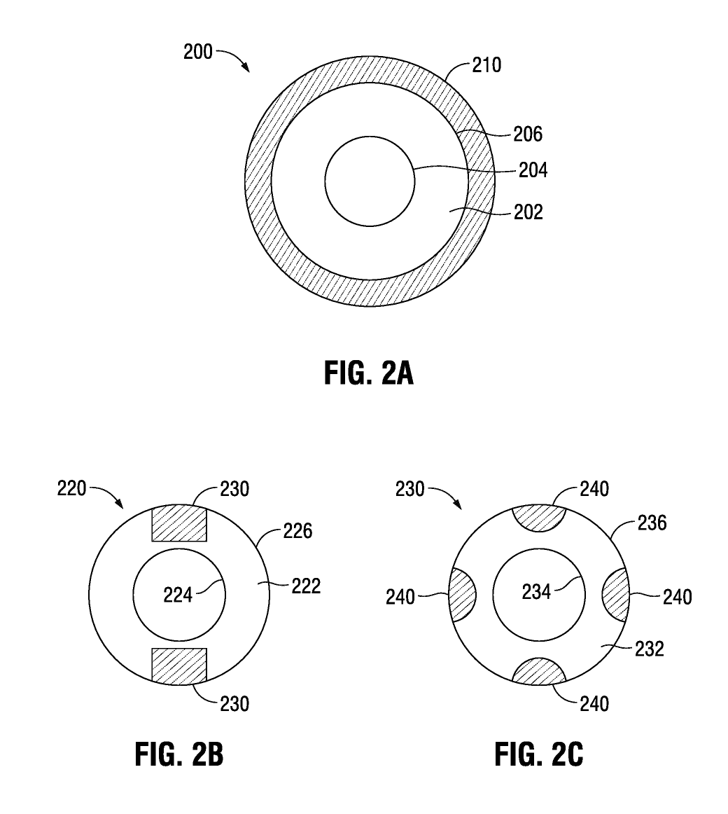

Various particular examples will be described. With additional reference to FIG. 2A, an example of a first tubing portion 202 coupled with a second tubing portion 210 to form a tube 200 will be described. In this example, for the formation of tube 200, the second tubing portion 210 may be located adjacent to the outer diameter 206 of the first tubing portion 202. First tubing portion 202 has an inner diameter 204 forming the interior tube for the fluid. The second tubing portion 210 may extend longitudinally in a continuous or intermittent manner with the outer diameter 206 of the first tubing portion. In this example, the second tubing portion 210 may be approximately circular shaped.

As another example, with additional reference to FIG. 2B, an example of a first tubing portion 222 coupled with a second tubing portion 230 to form a tube 220 will be described. In this example, the second tubing portion 230 may include two approximately rectangular shaped sections that may be located adjacent to the outer diameter 226 of the first tubing portion 222. These approximately rectangular shaped sections of the second tubing portion 230 may extend longitudinally in a continuous or interment manner with the outer diameter 226 of the first tubing portion 222 and may extend parallel to one another. First tubing portion 222 may have an inner diameter 224 forming the interior tube for the fluid.

As another example, with additional reference to FIG. 2C, an example of multiple sections of a second tubing portion 240 coupled to a first tubing portion 232 to form a tube 230 will be described. In this example, the multiple sections of the second tubing portion 240 are approximately cylindrical-shaped and are located adjacent to the outer diameter 236 of the first tubing portion 232. These approximately cylindrical-shaped tubing sections of the second tubing portion 240 may extend longitudinally in a continuous or intermittent matter with the outer diameter 236 of the first tubing portion 232 and may extend parallel to one another. First tubing portion 232 may have an inner diameter 234 forming the interior tube for the fluid.

In these particular examples of FIGS. 2A-2C, the second tubing portions may be of a harder PVC material (e.g., approximately 60 D) than the softer first tubing portions that may be a softer PVC material (e.g., approximately 92 A). By utilizing this type of implementation, in which the second tubing portions have a tensile modulus that is greater than the tensile modulus of the first tubing portions, the stiffness of the overall tubing increases and the natural frequency of the overall tubing increases resulting in a higher fidelity pressure monitoring signal, while maintaining suitable tubing flexibility to that of current pressure monitoring tubing. Therefore, the natural frequency increases, allowing for longer tubing, while maintaining tubing flexibility, allowing for ease of use by health care professionals.

It should be appreciated that FIGS. 2A-2C are purely examples of second tubing portions that may be located adjacent to the outer diameter of the first tubing portion that extend longitudinally in a continuous or intermittent manner with the outer diameter of the first tubing portion to form the tube. It should be appreciated that the second tubing portions may be of any suitable shape or size or configuration and that these are merely examples.

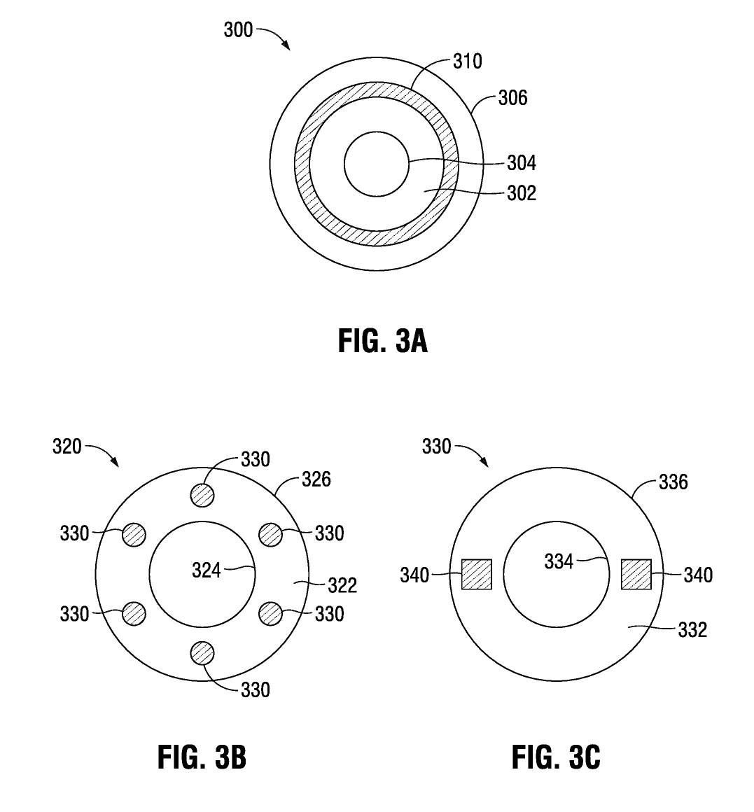

With additional reference to FIG. 3A, an example of first tubing portion 302 coupled with a second tubing portion 310 to form a tube 300 will be described. In this example, for the formation of tube 300, second tubing portion 310 is located between the outer diameter 306 and the inner diameter 304 of the first tubing portion 302. First tubing portion 302 has an inner diameter 304 forming the interior tube for the fluid. In this example, the second tubing portion 310 may extend longitudinally in a continuous or intermittent manner with the outer and inner diameters 306 and 304 of the first tubing portion 302. In this example the second tubing portion 310 may be approximately circular shaped.

As another example, with additional reference to FIG. 3B, an example of a plurality of approximately circular shaped sections of a second tubing portion 330 may be located between the outer diameter 326 and the inner diameter 324 of a first tubing portion 322 to form a tube 320. These approximately circular shaped sections of the second tubing portion 330 may extend longitudinally in a continuous or intermittent manner with the outer and inner diameter 326 and 324 of the first tubing portion 322 and may extend parallel to one another. First tubing portion 322 may have an inner diameter 324 forming the interior tube for the fluid.

As another example, with additional reference to FIG. 3C, an example of a pair of approximately rectangular shaped sections of a second tubing portion 340 may be located between the outer diameter 336 and the inner diameter 334 of a first tubing portion 332 to form a tube 330. These approximately rectangular shaped sections of the second tubing portions 340 may extend longitudinally in a continuous or intermittent manner with the outer and inner diameter 336 and 334 of the first tubing portion 332 and may extend parallel to one another. First tubing portion 332 may have an inner diameter 334 forming the interior tube for the fluid.

It should be appreciated that FIGS. 3A-3C are purely examples of second tubing portions that may be located between the outer diameter and the inner diameter of the first tubing portion and that extend longitudinally in a continuous or intermittent manner with the outer and inner diameter of the first tubing portion to form the tube. It should be appreciated that the second tubing portions may be of any suitable shape or size or configuration and that these are merely examples.

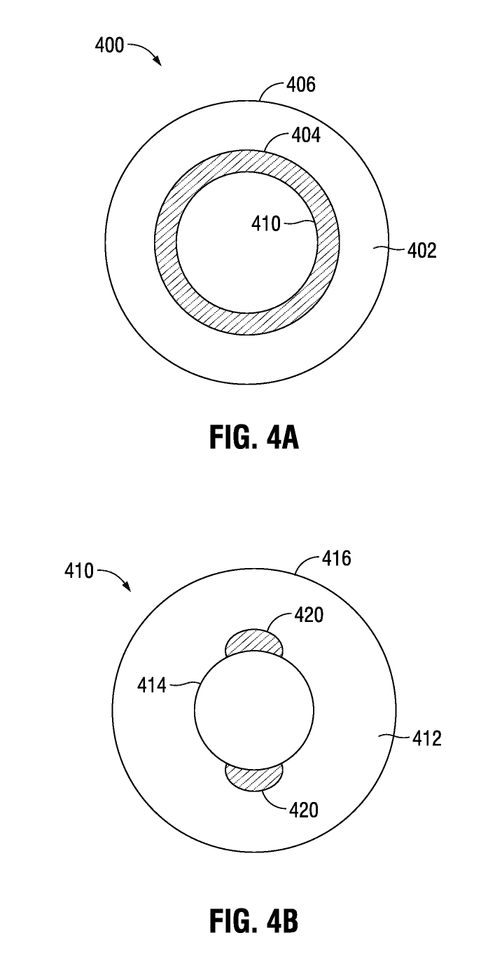

With additional reference to FIG. 4A, an example of a first tubing portion 402 coupled with a second tubing portion 410 to form a tube 400 will be described. In this example, for the formation of tube 400, the second tubing portion 410 may be located adjacent to the inner diameter 404 of the first tubing portion 402. The second tubing portion 410 may extend longitudinally in a continuous or intermittent manner with the inner diameter 404 of the first tubing portion 402. In this example the second tubing portion 410 may be approximately circular shaped. The second tubing portion 410 may form the interior tube for the fluid.

As another example, with additional reference to FIG. 4B, an example of a first tubing portion 412 coupled with a second tubing portion 420 to form a tube 410 will be described. In this example, the second tubing portion 420 may include at least two approximately cylindrical-shaped sections 420 that are located adjacent to the inner diameter 414 of the first tubing portion 412 and that extend longitudinally in a continuous or intermittent manner with the inner diameter 414 of the first tubing portion 412 and may extend parallel to one another. First tubing portion 412 may have an inner diameter 414 that along with the second tubing portions 420 may form the interior tube for the fluid.

It should be appreciated that the second tubing portions presented in FIGS. 2-4 are merely examples of second tubing portions and that the second tubing portions may be of any suitable shape or size or configuration and that these merely examples. Further, it should be appreciated that all of the previously described first and second tubing portions may be coupled together by utilizing a co-extrusion process. As part of the co-extrusion process, the tubing portions may be over molded, heated until merged/melted together, or held in place adjacent to one another with the aid of a tertiary component and shrink-wrapped. As has been previously described the first and second tubing portions my comprise polyvinyl chloride (PVC) material. However, in other embodiments, the first tubing portion may comprise a PVC material or an ethylene vinyl acetate (EVA)) whereas the second tubing portion may comprise at least one of metallic wires, metallic braids, aramid fibers, glass fibers, plastic fibers, fluid, or any suitable stiffer material.

As has been described, embodiments of the invention may relate to a two part co-extruded tube whose primary material (e.g., a polyvinyl chloride (PVC) or ethylene vinyl acetate (EVA)) may have a first tensile modulus (e.g., relatively low—such as hardness approximately between 80 A-110 A), and may further utilize a secondary material that has a significantly higher tensile modulus (e.g., a hardness approximately 60 D). In the previously described examples of FIGS. 2-4, the second tubing portions may be of a harder material (e.g., approximately 60 D) than the softer first tubing portions that may be a softer material (e.g., approximately between 80 A-110 A). By utilizing this type of implementation, in which the second tubing portions have a tensile modulus that is greater than the tensile modulus of the first tubing portions, the stiffness of the overall tubing increases and the natural frequency of the overall tubing increases resulting in a higher fidelity pressure monitoring signal, while maintaining suitable tubing flexibility to that of current pressure monitoring tubing. Therefore, the natural frequency increases, allowing for longer tubing, while maintaining tubing flexibility, allowing for ease of use by health care professionals.

Additional embodiments of the invention will be hereafter described. These additional embodiments may likewise relate to a co-extruded tube, such as tube 109, that may have a primary material (e.g., a polyvinyl chloride (PVC) or ethylene vinyl acetate (EVA)) that may have first tensile modulus (e.g., relatively low—such as a hardness approximately 80 A-92 A), and that may further utilize a secondary material that has a significantly higher tensile modulus (e.g., a hardness approximately 60 D-70 D). Multiple fluid paths may be formed within the primary material of the tube 109 to provide multiple fluid paths between the measurement site of the patient and the pressure transducer, as will be described in more detail hereafter. The secondary material may be co-extruded within the primary material of the tube 109 and may be routed longitudinally within the tubing of tube, forming veins of stiffening material. These veins may be positioned so as not to significantly increase the bending modulus of the tube 109, but may increase the tensile modulus of the tube. By increasing the tubing tensile modulus in this way, the stiffness of tube 109 increases and the natural frequency of tube increases, resulting in a higher fidelity pressure monitoring signal, while maintaining equivalent tubing flexibility to that of current pressure monitoring tubing. Therefore, the natural frequency increases, allowing for longer tubing, while maintaining tubing flexibility, allowing for ease of use by health care professionals.

As has been described with reference to FIG. 1, a pressure monitoring system 100 may include a measurement site 108, a tube 109, and a pressure transducer 115 that may be held by a sensor holding apparatus 112. Tube 109 may be coupled between the measurement site 108 of the patient and the pressure transducer 115. As has been described, the pressure signal is generated and transmitted from the measurement site 108 (e.g., vein, artery, etc.) via the catheter and the fluid-filled tube 109, as fluid pressure to the pressure transducer (PT) 115, where it may be converted to an electrical pressure signal and outputted to the patient monitor 107. Thus, tube 109 may contain a fluid for pressure measurement by the pressure transducer 115 from the measurement site 108. In particular, in one embodiment, as will be described in more detail hereafter, multiple fluid paths may be formed within the primary material of the tube 109, such that, the tube may contain multiple fluid paths to contain fluid for pressure measurement by the pressure transducer 115 from the measurement site 108.

As will be described, in one embodiment, tube 109 may include a first tubing portion that includes an outer diameter and a plurality of inner diameters. The first tubing portion has a first tensile modulus. The plurality of inner diameters of the first tubing portion may be used to form a plurality of fluid paths to contain fluid used for pressure measurement by the pressure transducer 115 from the measurement site 118. Further, the tube 109 may include a second tubing portion. The first and second tubing portions may be coupled together to form the tube 109. The second tubing portion may extend longitudinally with the first tubing portion in a continuous or intermittent manner. The second tubing portion may have a second tensile modulus that is greater than the first tensile modulus of the first tubing portion.

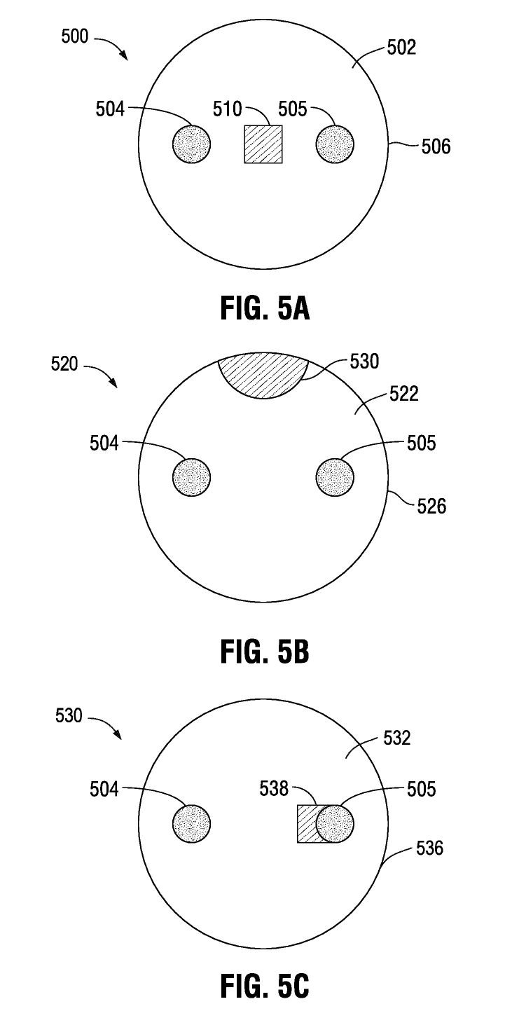

Various particular examples will be hereafter described. With additional reference to FIG. 5A, an example of a first tubing portion 502 coupled with a second tubing portion 510 to form a tube 500 will be described. In this example, first tubing portion 502 includes a plurality of inner diameters 504 and 505 to form a plurality of fluid paths or interior tubes to contain fluid for measurement by the pressure transducer from the measurement site. Further, in this example, the second tubing portion 510 is located between the outer diameter 506 and the inner diameters 504 and 505 of the first tubing portion 502 that form the plurality of fluid paths. The second tubing portion 510 may extend longitudinally in a continuous or intermittent manner with the outer diameter 506 of the first tubing portion 502. In this example, the second tubing portion 510 may be approximately square or rectangular shaped.

As another example, with additional reference to FIG. 5B, an example of a first tubing portion 522 coupled with a second tubing portion 530 to form a tube 520 will be described. In this example, first tubing portion 522 includes a plurality of inner diameters 504 and 505 to form a plurality of fluid paths or interior tubes to contain fluid for measurement by the pressure transducer from the measurement site. Further, in this example, the second tubing portion 530 is located adjacent to the outer diameter 526 of the first tubing portion. The second tubing portion 530 may extend longitudinally in a continuous or intermittent manner with the outer diameter 526 of the first tubing portion 522. In this example, the second tubing portion 530 may be approximately cylindrical-shaped.

With additional reference to FIG. 5C, an example of a first tubing portion 532 coupled with a second tubing portion 538 to form a tube 530 will be described. In this example, first tubing portion 532 includes a plurality of inner diameters 504 and 505 to form a plurality of fluid paths or interior tubes to contain fluid for measurement by the pressure transducer from the measurement site. Further, in this example, the second tubing portion 538 is located adjacent to inner diameter 505 of the first tubing portion 532. The second tubing portion 538 may extend longitudinally in a continuous or intermittent manner with the outer diameter 536 of the first tubing portion 532. In this example, the second tubing portion 538 may be approximately square or rectangular shaped with an interior circular section that abuts the inner diameter of interior tube 505 that forms one of the fluid paths.

Further, as will be described in more detail hereafter, other embodiments may include a second tubing portion that includes a plurality of second tubing portions (e.g., two or more tubing portions), in which, the second tubing portions may be: located between the outer diameter of the first tubing portion and the inner diameters of the first tubing portion; located adjacent to the outer diameter of the first tubing portion; or located adjacent to the inner diameters of the first tubing portion. In any of these instances, the second tubing portions may extend longitudinally in a continuous or intermittent manner with the outer diameter of the first tubing portion.

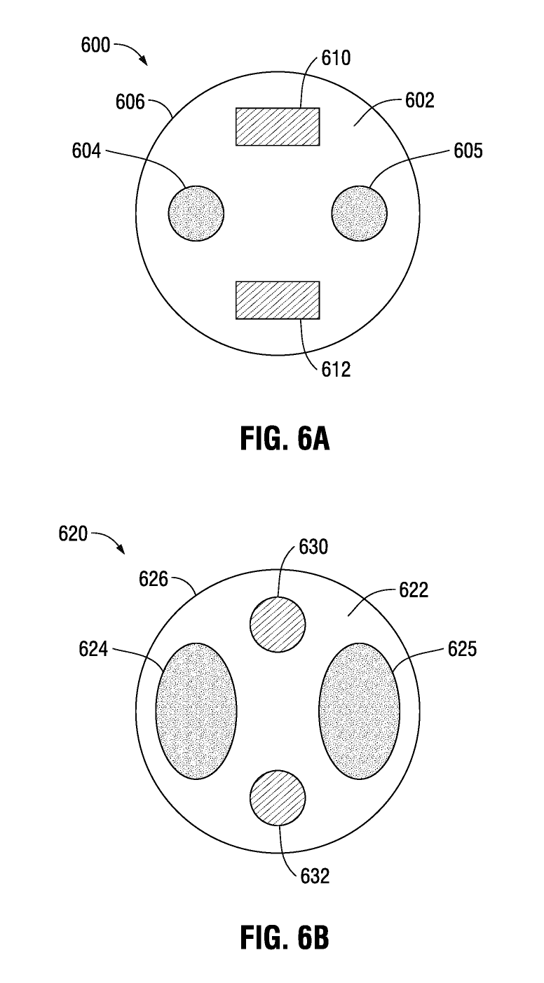

With additional reference to FIG. 6A, an example of a first tubing portion 602 coupled with a pair of aligned second tubing portions 610 and 612 to form a tube 600 will be described. In this example, first tubing portion 602 includes a plurality of inner diameters 604 and 605 to form a plurality of fluid paths or interior tubes to contain fluid for measurement by the pressure transducer from the measurement site. Further, in this example, the second tubing portions 610 and 612 are located between the outer diameter 606 and the inner diameters 604 and 605 of the first tubing portion 602 that form the plurality of fluid paths. The second tubing portions 610 and 612 may extend longitudinally in a continuous or intermittent manner with the outer diameter 606 of the first tubing portion 602. In this example, the second tubing portions 610 and 612 may be approximately square or rectangular shaped.

With additional reference to FIG. 6B, an example of a first tubing portion 622 coupled with a pair of aligned second tubing portions 630 and 632 to form a tube 620 will be described. In this example, first tubing portion 622 includes a plurality of inner elongated diameters 624 and 625 to form a plurality of fluid paths or interior tubes to contain fluid for measurement by the pressure transducer from the measurement site. Further, in this example, the second tubing portions 630 and 632 are located between the outer diameter 626 and the inner elongated diameters 624 and 624 of the first tubing portion 622 that form the plurality of fluid paths. The second tubing portions 630 and 632 may extend longitudinally in a continuous or intermittent manner with the outer diameter 626 of the first tubing portion 622. In this example, the second tubing portions 630 and 632 may be approximately circular or cylindrically shaped. Also, in this example, the inner diameters 624 and 625 to form the fluid paths or interior tubes may be approximately cylindrically shaped (e.g., elongated), elliptical shaped, etc., as opposed to more closely circular shaped, as the previously described inner diameters. However, it should be appreciated, that the exact shape of the inner diameter may be a design choice, any shape may be suitable, as will be described in more detail hereafter.

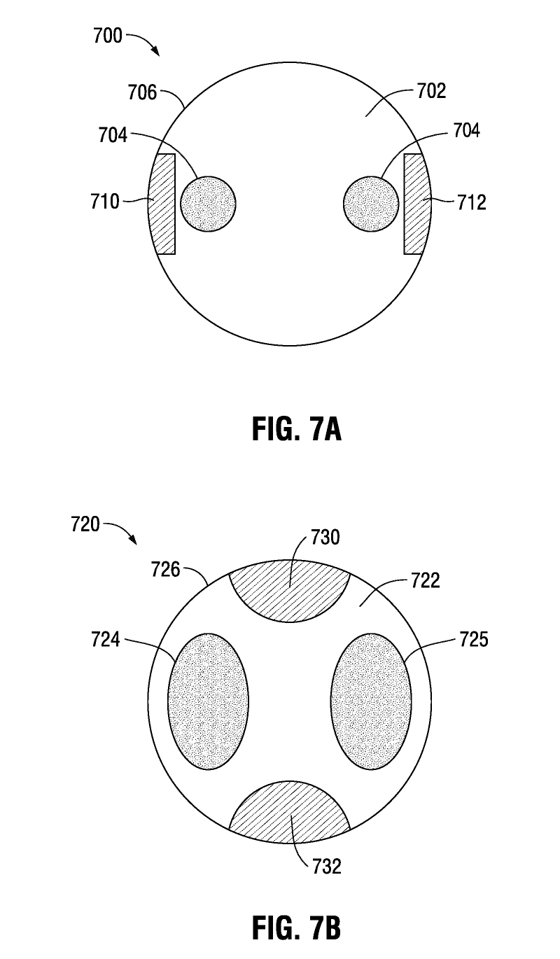

With additional reference to FIG. 7A, an example of a first tubing portion 702 coupled with a pair of aligned second tubing portions 710 and 712 to form a tube 700 will be described. In this example, first tubing portion 702 includes a plurality of inner diameters 704 and 705 to form a plurality of fluid paths or interior tubes to contain fluid for measurement by the pressure transducer from the measurement site. Further, in this example, the second tubing portions 710 and 712 are located adjacent to the outer diameter 706 of the first tubing portion 710. The second tubing portions 710 and 712 may extend longitudinally in a continuous or intermittent manner with the outer diameter 706 of the first tubing portion 702. In this example, the second tubing portions 710 and 712 may be approximately square or rectangular shaped with an exterior circular shape to mate with the outside diameter 706.

With additional reference to FIG. 7B, an example of a first tubing portion 722 coupled with a pair of aligned second tubing portions 730 and 732 to form a tube 720 will be described. In this example, first tubing portion 722 includes a plurality of inner elongated diameters 724 and 725 to form a plurality of fluid paths or interior tubes to contain fluid for measurement by the pressure transducer from the measurement site. Further, in this example, the second tubing portions 730 and 732 are located adjacent to the outer diameter 726 of the first tubing portion 722. The second tubing portions 730 and 732 may extend longitudinally in a continuous or intermittent manner with the outer diameter 726 of the first tubing portion 722. In this example, the second tubing portions 730 and 732 may be approximately cylindrically shaped with an exterior circular shape to mate with the outside diameter 726.

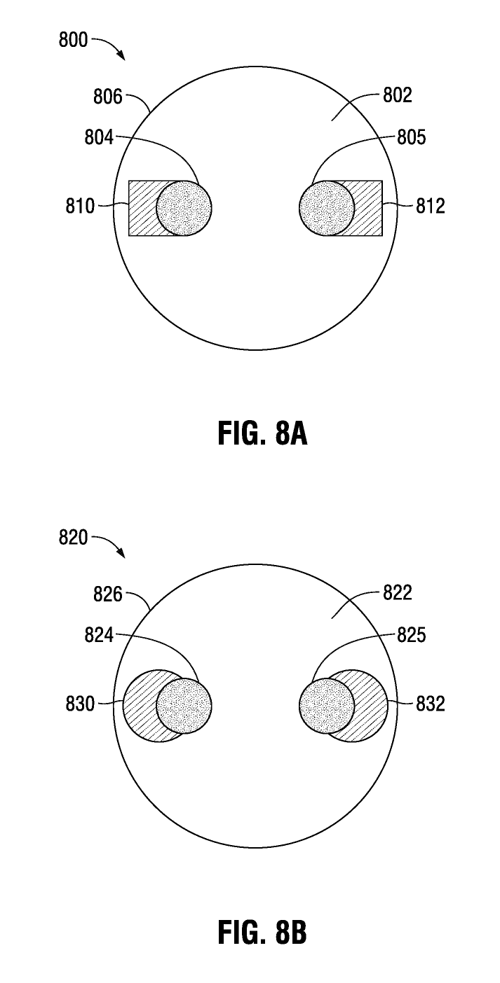

With additional reference to FIG. 8A, an example of a first tubing portion 802 coupled with a pair of aligned second tubing portions 810 and 812 to form a tube 800 will be described. In this example, first tubing portion 802 includes a plurality of inner diameters 804 and 805 to form a plurality of fluid paths or interior tubes to contain fluid for measurement by the pressure transducer from the measurement site. Further, in this example, the second tubing portions 810 and 812 are located adjacent to the inner diameters 810 and 812 of the first tubing portion 810, respectively. The second tubing portions 810 and 812 may extend longitudinally in a continuous or intermittent manner with the outer diameter 806 of the first tubing portion 802. In this example, the second tubing portions 810 and 812 may be approximately square or rectangular shaped with an exterior circular shape to mate with the inside diameters 804 and 805, respectively.

With additional reference to FIG. 8B, an example of a first tubing portion 822 coupled with a pair of aligned second tubing portions 830 and 832 to form a tube 820 will be described. In this example, first tubing portion 822 includes a plurality of inner diameters 824 and 825 to form a plurality of fluid paths or interior tubes to contain fluid for measurement by the pressure transducer from the measurement site. Further, in this example, the second tubing portions 830 and 832 are located adjacent to the inner diameters 824 and 825 of the first tubing portion 822, respectively. The second tubing portions 830 and 832 may extend longitudinally in a continuous or intermittent manner with the outer diameter 826 of the first tubing portion 822. In this example, the second tubing portions 830 and 832 may be approximately cylindrically shaped with an exterior circular shape to mate with the inside diameters 824 and 825, respectively.

In these particular examples of FIGS. 5A-5C, 6A-6B, 7A-7B, and 8A-8B, the second tubing portions may be of a harder material (e.g., PVC, approximately 60 D-70 D) than the softer first tubing portions that may be a softer material (e.g., PVC, approximately 80 A-92 A). By utilizing this type of implementation, in which the second tubing portions have a tensile modulus that is greater than the tensile modulus of the first tubing portions, the stiffness of the overall tubing increases and the natural frequency of the overall tubing increases resulting in a higher fidelity pressure monitoring signal, while maintaining suitable tubing flexibility to that of current pressure monitoring tubing. Therefore, the natural frequency increases, allowing for longer tubing, while maintaining tubing flexibility, allowing for ease of use by health care professionals.

It should be appreciated that FIGS. 5A-5C, 6A-6B, 7A-7B, and 8A-8B, are purely examples of various first tubing portions having various inner diameters to form fluid paths or interior tubes and various second tubing portions that may be located adjacent to the outer diameter of the first tubing portion, located between the outer diameter and the inner diameters of the first tubing portion, or located adjacent to the inner diameters of the first tubing portion. Although various shapes, sizes, and configurations have been shown of first and second tubing portions, it should be appreciated that any suitable configuration of first tubing portions (e.g., to form any suitable number of fluid paths) and second tubing portions (e.g., to form any suitable number of higher tensile portions) may be utilized and that the first and second tubing portions may be of any suitable shapes or sizes, and that these are merely examples.

Further, it should be appreciated that all of the previously described first and second tubing portions may be coupled together by utilizing a co-extrusion process. As part of the co-extrusion process, the tubing portions may be over molded, heated until merged/melted together, or held in place adjacent to one another with the aid of a tertiary component and shrink-wrapped. As has been previously described the first and second tubing portions my comprise polyvinyl chloride (PVC) material. However, in other embodiments, the first tubing portion may comprise a PVC material or EVA material whereas the second tubing portion may comprise at least one of metallic wires, metallic braids, aramid fibers, glass fibers, plastic fibers, fluid, or any suitable stiffer material.

As has been described, embodiments of the invention may relate to a two part co-extruded tube whose primary material ((e.g., a PVC or EVA may have a first tensile modulus (e.g., relatively low—such as hardness approximately between 80 A-92 A), and may further utilize a secondary material that has a significantly higher tensile modulus (e.g., a hardness approximately 60 D-70 D). In the previously described examples of FIGS. 5-8, the second tubing portions may be of a harder material (e.g., approximately 60 D-70 D) than the softer first tubing portions that may be a softer material (e.g., approximately between 80A-92 A). By utilizing this type of implementation, in which the second tubing portions have a tensile modulus that is greater than the tensile modulus of the first tubing portions, the stiffness of the overall tubing increases and the natural frequency of the overall tubing increases resulting in a higher fidelity pressure monitoring signal, while maintaining suitable tubing flexibility to that of current pressure monitoring tubing. Therefore, the natural frequency increases, allowing for longer tubing, while maintaining tubing flexibility, allowing for ease of use by health care professionals.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A pressure monitoring system for a patient including a measurement site, a tube, and a pressure transducer, the tube coupled between the measurement site of the patient and the pressure transducer, the tube containing a fluid for pressure measurement by the pressure transducer from the measurement site, the tube comprising:
a first tubing portion including an outer diameter and a plurality of inner diameters, the first tubing portion having a first tensile modulus, the plurality of inner diameters of the first tubing portion to form a plurality of fluid paths to contain the fluid; and
a second tubing portion, the first and second tubing portions being coupled together to form the tube, the second tubing portion extending longitudinally with the first tubing portion in a continuous or intermittent manner, the second tubing portion having a second tensile modulus that is greater than the first tensile modulus of the first tubing portion.

2. The pressure monitoring system of claim 1, wherein the second tubing portion is located between the outer diameter and the inner diameters of the first tubing portion that form the plurality of fluid paths.

3. The pressure monitoring system of claim 1, wherein the second tubing portion is located adjacent to the outer diameter of the first tubing portion.

4. The pressure monitoring system of claim 1, wherein the second tubing portion is located adjacent to at least one of the inner diameters of the first tubing portion that form the plurality of fluid sections.

5. The pressure monitoring system of claim 1, wherein, the second tubing portion includes a plurality of second tubing portions located between the outer diameter of the first tubing portion and the inner diameters of the first tubing portion or the second tubing portion includes a plurality of second tubing portions located adjacent to the outer diameter of the first tubing portion.

6. The pressure monitoring system of claim 1, wherein the measurement site includes a catheter connected to one of a vein or artery of the patient.

7. The pressure monitoring system of claim 1, wherein the first tubing portion and the second tubing portion are coupled together through a co-extrusion process, over molded, and heated until the first tubing portion and the second tubing portion are merged/melted together or are held in place adjacent to one another with the aid of a tertiary component and shrink-wrapped.

8. The pressure monitoring system of claim 1, wherein the first and second tubing portions comprise polyvinyl chloride material.

9. The pressure monitoring system of claim 1, wherein the first tubing portion comprises a polyvinyl chloride material and the second tubing portion comprises at least one of metallic wires, metallic braids, aramid fibers, glass fibers, plastic fibers, or fluid.

10. A pressure monitoring system for a patient comprising:
a measurement site;
a tube; and
a pressure transducer, wherein the tube is coupled between the measurement site of the patient and the pressure transducer, and the tube contains a fluid for pressure measurement by the pressure transducer from the measurement site, the tube comprising:
a first tubing portion including an outer diameter and a plurality of inner diameters, the first tubing portion having a first tensile modulus, the plurality of inner diameters of the first tubing portion to form a plurality of fluid paths to contain the fluid; and
a second tubing portion, the first and second tubing portions being coupled together to form the tube, the second tubing portion extending longitudinally with the first tubing portion in a continuous or intermittent manner, the second tubing portion having a second tensile modulus that is greater than the first tensile modulus of the first tubing portion.

11. The pressure monitoring system of claim 10, wherein the second tubing portion is located between the outer diameter and the inner diameters of the first tubing portion that form the plurality of fluid paths.

12. The pressure monitoring system of claim 10, wherein the second tubing portion is located adjacent to the outer diameter of the first tubing portion.

13. The pressure monitoring system of claim 10, wherein the second tubing portion is located adjacent to at least one of the inner diameters of the first tubing portion that form the plurality of fluid sections.

14. The pressure monitoring system of claim 10, wherein, the second tubing portion includes a plurality of second tubing portions located between the outer diameter of the first tubing portion and the inner diameters of the first tubing portion or the second tubing portion includes a plurality of second tubing portions located adjacent to the outer diameter of the first tubing portion.

15. The pressure monitoring system of claim 10, wherein the measurement site includes a catheter connected to one of a vein or artery of the patient.

16. The pressure monitoring system of claim 10, wherein the first tubing portion and the second tubing portion are coupled together through a co-extrusion process, over molded, and heated until the first tubing portion and the second tubing portion are merged/melted together or are held in place adjacent to one another with the aid of a tertiary component and shrink-wrapped.

17. The pressure monitoring system of claim 10, wherein the first and second tubing portions comprise polyvinyl chloride material.

18. The pressure monitoring system of claim 10, wherein the first tubing portion comprises a polyvinyl chloride material and the second tubing portion comprises at least one of metallic wires, metallic braids, aramid fibers, glass fibers, plastic fibers, or fluid.

19. A method for displaying pressure measurements of a patient comprising:
coupling a tube between a measurement site of the patient and a pressure transducer, the tube containing a fluid for pressure measurement by the pressure transducer from the measurement site, the tube comprising:
a first tubing portion including an outer diameter and a plurality of inner diameters, the first tubing portion having a first tensile modulus, the plurality of inner diameters of the first tubing portion to form a plurality of fluid paths to contain the fluid; and
a second tubing portion, the first and second tubing portions being coupled together to form the tube, the second tubing portion extending longitudinally with the first tubing portion in a continuous or intermittent manner, the second tubing portion having a second tensile modulus that is greater than the first tensile modulus of the first tubing portion; and
displaying the pressure measurement of the patient on a patient monitor.

20. The method of claim 19, wherein the second tubing portion is located between the outer diameter and the inner diameters of the first tubing portion that form the plurality of fluid paths.

21. The method of claim 19, wherein the second tubing portion is located adjacent to the outer diameter of the first tubing portion.

22. The method of claim 19, wherein the second tubing portion is located adjacent to at least one of the inner diameters of the first tubing portion that form the plurality of fluid sections.

23. The method of claim 19, wherein, the second tubing portion includes a plurality of second tubing portions located between the outer diameter of the first tubing portion and the inner diameters of the first tubing portion or the second tubing portion includes a plurality of second tubing portions located adjacent to the outer diameter of the first tubing portion.

24. The method of claim 19, wherein the measurement site includes a catheter connected to one of a vein or artery of the patient.

25. The method of claim 19, wherein the first tubing portion and the second tubing portion are coupled together through a co-extrusion process, over molded, and heated until the first tubing portion and the second tubing portion are merged/melted together or are held in place adjacent to one another with the aid of a tertiary component and shrink-wrapped.

26. The method of claim 19, wherein the first and second tubing portions comprise polyvinyl chloride material.

27. The method of claim 19, wherein the first tubing portion comprises a polyvinyl chloride material and the second tubing portion comprises at least one of metallic wires, metallic braids, aramid fibers, glass fibers, plastic fibers, or fluid.
